(19) United States
(12) Patent Application Publication
(10) Pub. No.: US 2025/0217976 A1
(43) Pub. Date: Jul. 3, 2025

(54) SYSTEM AND METHOD OF MEDICAL IMAGE QUALITY FEEDBACK

(71) Applicant: AJOU UNIVERSITY INDUSTRY-ACADEMIC COOPERATION FOUNDATION, Suwon-si (KR)

(72) Inventors: Jung Won Lee, Seoul (KR); Dong Yeon Yoo, Suwon-si (KR); Hyeon-jin Choi, Hwaseong-si (KR)

(21) Appl. No.: 18/999,698

(22) Filed: Dec. 23, 2024

(30) Foreign Application Priority Data
Dec. 28, 2023 (KR) 10-2023-0195700

(51) Int. Cl. G06T 7/00; G16H 30/40; G16H 80/00

(52) U.S. Cl. CPC G06T 7/0012; G16H 30/40; G16H 80/00; G06T 2207/10116; G06T 2207/20081; G06T 2207/30061; G06T 2207/30168

(57) ABSTRACT

A method for feeding back medical image quality is disclosed. The medical image quality feedback method according to the present disclosure includes receiving X-ray image data; extracting a region of interest to be diagnosed from the image data; evaluating a quality of the region of interest depending on a predetermined criterion; and generating feedback information including whether to request rephotographing based on a result of the quality evaluation.

### CROSS-REFERENCE TO RELATED APPLICATION

This application claims priority to and the benefit of Korean Patent Application No. 10-2023-0195700, filed on Dec. 28, 2023, the disclosures of which is incorporated herein by reference in its entirety.

### TECHNICAL FIELD

The present disclosure relates to a system and method for feeding back medical image quality.

### RELATED ART

Chest images taken by X-ray photography are used to diagnose chest diseases. Diagnosis by X-ray images generally consists of a step in which a radiologist takes photographs using an X-ray equipment, transmits the photographed images to a doctor's terminal, and then the doctor looks at them and makes a diagnosis.

The chest images taken by X-ray photography are convenient, but there are variables related to image quality, which may affect diagnosis. For example, if the patient does not inhale sufficiently when taking the photograph, the lower part of the lungs may not be clearly seen, and lesions may be missed.

To solve the above deficiencies, Patent Publication KR-10-2021-7000146 proposes a technique to automatically take photographs at the time of maximum inhalation by recognizing the patient's respiratory phase. However, it may not be determined that the maximum inhalation point is always suitable for diagnosis or that only the maximum inhalation point is suitable for diagnosis.

The disclosure of this section is to provide background information relating to the present disclosure. Applicant does not admit that any information contained in this section constitutes prior art.

### SUMMARY

In the radiology department, factors that make it difficult to diagnose the chest are separately defined as above, and the quality of the image is evaluated based on this, and if it is determined to be unsuitable for diagnosis, additional photographing procedures are performed. That is, if it is determined that the quality of the image is not suitable for diagnosis, the diagnosis is not performed. However, the additional photographing is a very cumbersome and difficult procedure for patients who have difficulty in moving in the clinical field, and it is practically difficult to evaluate the images taken in real time due to the limitation of the number of radiologists.

Some aspects of the present disclosure provide a system and method for feeding back medical image quality that determine whether an image is suitable for diagnosis based on a photographed image and to provide immediate feedback.

A medical image quality feedback method according to an embodiment of the present disclosure may be performed on at least one processor and include receiving X-ray image data; extracting a region of interest to be diagnosed from the image data; evaluating a quality of the region of interest depending on a predetermined criterion; and generating feedback information including whether to request rephotographing based on a result of the quality evaluation.

In an embodiment of the present disclosure, the feedback information may include a reason for not meeting the quality criterion.

In an embodiment of the present disclosure, the reason for not meeting the quality standard may include at least one of whether the criterion for evaluating a structure arrangement is met and whether the criterion for evaluating an interior of a structure is met.

In an embodiment of the present disclosure, the evaluation of the structure arrangement may include an evaluation of at least one of an evaluation of coverage range, an evaluation of bilateral symmetry, and an evaluation of scapular position.

In an embodiment of the present disclosure, the evaluation of the interior of the structure may include an evaluation of at least one of an evaluation of a degree of inhalation, an evaluation of a penetration state and a resolution, and an evaluation of a presence or absence of artificial shadings.

In an embodiment of the present disclosure, the medical image quality feedback system may further include setting a criterion of the quality evaluation; and determining learning data depending on the quality evaluation criterion and performing machine learning, wherein the predetermined criterion is the quality evaluation criterion, the quality evaluation is output by the machine-learned result, the region of interest is a lung area, and the quality evaluation may include at least one of an evaluation of a structure arrangement and an evaluation of an interior of a structure.

In an embodiment of the present disclosure, the medical image quality feedback system may further include setting a criterion of the quality evaluation; determining learning data depending on the quality evaluation criterion and performing machine learning; determining the X-ray image as an image to be transmitted to an expert and transmitting it to an expert terminal; receiving expert feedback information from the expert terminal; and determining the expert feedback information as additional learning data and performing machine learning, wherein the quality evaluation may be output by the machine-learned result.

The medical image quality feedback system according to an embodiment of the present disclosure may include a communicator configured to receive X-ray image data; and a processor configured to extract a region of interest to be diagnosed from the image data, evaluate a quality of the region of interest depending on a predetermined criterion, and generate feedback information including whether to request rephotographing based on a result of the quality evaluation.

According to the present disclosure, the system determines whether the image is suitable for diagnosis based on the photographed image and provides immediate feedback to immediately re-photograph the image when the image quality criterion is not met, thereby eliminating the inconvenience of repeating the re-photographing procedure in the hospital (e.g., healthcare provider or healthcare provider entity, and the like).

### BRIEF DESCRIPTION OF THE DRAWINGS

FIG. 1 shows a medical image quality feedback system according to an embodiment of the present disclosure.

FIG. 2 shows a medical image quality feedback method according to an embodiment of the present disclosure.

FIG. 3 show in detail some configurations of a medical image quality feedback method according to an embodiment of the present disclosure.

FIG. 4 show in detail some configurations of a medical image quality feedback method according to an embodiment of the present disclosure.

FIG. 5 visually shows a process of extracting a lung area according to an embodiment of the present disclosure.

FIG. 6 visually shows an embodiment of an evaluation of a structure arrangement according to an embodiment of the present disclosure.

FIG. 7 visually shows an evaluation of an interior of a structure according to an embodiment of the present disclosure.

FIG. 8 shows together X-ray image information and feedback information according to an embodiment of the present disclosure.

### DETAILED DESCRIPTION

The present disclosure may be subjected to various transformations and have various embodiments, and specific embodiments are illustrated in the drawings and will be described in detail in the detailed description. However, this is not intended to limit the present disclosure to specific embodiments, and it should be understood that it includes all conversions, equivalents, or alternatives included in the spirit and technical scope of the present disclosure.

In describing the present disclosure, a detailed description of the related known technology is omitted if it is judged that the gist of the present disclosure may obscure.

Hereinafter, embodiments of the present disclosure will be described in detail with reference to the accompanying drawings.

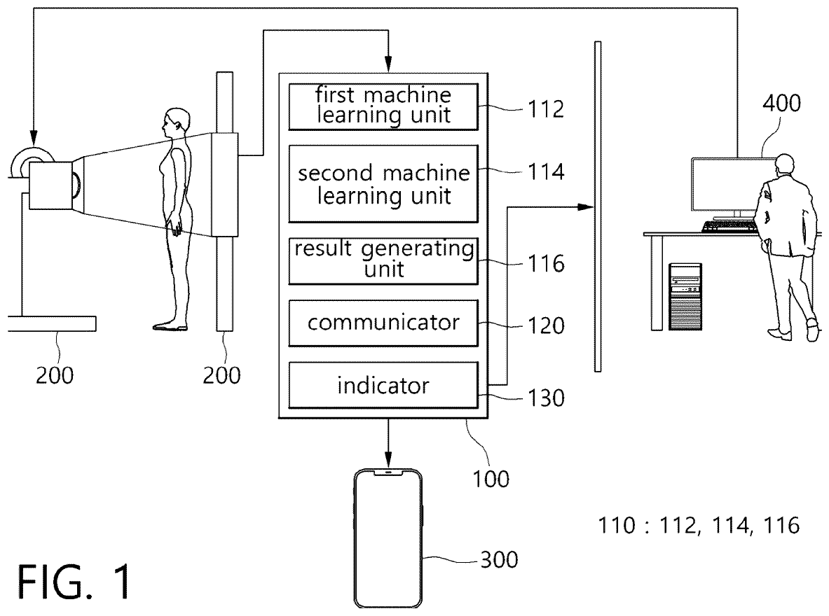

FIG. 1 shows a medical image quality feedback system 100 according to an embodiment of the present disclosure.

Referring to FIG. 1, the medical image quality feedback system 100 according to an embodiment of the present disclosure includes a controller 110, a communicator 120, and an indicator 130.

The controller 110 may be a server, a cloud server, or a terminal in which a processor is embedded. The controller 110 may include a plurality of terminals that are physically or logically separated to provide information processing, and may be distributed and installed in various places or may be provided in a form of using a cloud service for storing and processing information.

The controller 110 includes a first machine learning unit 112, a second machine learning unit 114, and a result generation unit 116.

The first machine learning unit 112 may precede machine learning by artificial intelligence and output a result of later input information based on the learned information.

The first machine learning unit 112 may extract a lung area by receiving an X-ray image. The X-ray image and the extracted lung area information may be provided in advance to the first machine learning unit 112 as pre-learning data.

The second machine learning unit 114 may precede machine learning by artificial intelligence and output a result of later input information based on the learned information.

The second machine learning unit 114 may evaluate the quality from a lung area image. The lung area image and quality evaluation result may be provided in advance to the learning unit as pre-learning data.

An evaluation information on the quality may include evaluation information on a structure arrangement and evaluation information on an interior of a structure.

The result generating unit 116 may generate quality result information corresponding to the quality evaluation result.

The quality result information may include information on whether the quality criterion is met, if not met, information on the reason for this, information on a correction request for a photographing posture corresponding to the above reason, and information on a rephotographing request.

The communicator 120 is connected to the controller 110.

The communicator 120 may transmit and receive information to and from an X-ray image acquisition device 200, the first user terminal 300, and the second user terminal 400 under the control of the controller 110.

The indicator 130 is connected to the controller 110.

The indicator 130 may output the feedback information generated under the control of the controller 110 in a form that can be visually or audibly recognized.

The indicator 130 may be, for example, at least one of a display means and a voice output means. The display means may be a display such as a monitor, and the voice output means may be a speaker built in or connected to the terminal.

For example, the indicator 130 may visually or audibly output information such as, “The right shoulder is lower than the left shoulder and is not balanced left and right, so it corrects the posture and prefers to retake it.”

The X-ray image acquisition device 200 may be connected to the image quality feedback system 100 through a wired/wireless network to transmit and receive information.

The X-ray image acquisition device 200 may be, for example, an X-ray photographing device.

The X-ray image acquisition device 200 may acquire X-ray image information and provide it to the image quality feedback system 100.

In embodiments, the first user terminal 300 may include a controller, a communicator, a storage, a display, and an input unit.

The first user terminal 300 may transmit and receive information to and from the image quality feedback system 100 through the communicator 120, and may store and execute applications that may be installed in the first user terminal 300 through the storage.

A first user who owns and/or uses the first user terminal 300 may be an administrator or a user of the X-ray image acquisition device 200, and the first user terminal 300 may be a desk top computer or a smartphone.

In embodiments, the second user terminal 400 may include a controller, a communicator, a storage, a display, and an input unit.

The second user terminal 400 may transmit and receive information to and from the image quality feedback system 100 through the communicator 120, and store and execute applications that may be installed in the second user terminal 400 through the storage.

A second user who owns or uses the second user terminal 400 may be a healthcare provider (e.g., doctor) who performs diagnosis using the X-ray image, and the healthcare provider may utilize the second user terminal 400 to transmit and receive information to and from the image quality feedback system 100 in accordance with the embodiment of the second user terminal 400, as disclosed herein.

In some examples, an administrator or a user of the X-ray image acquisition device 200 may also utilize the second user terminal 400 to transmit and receive information to and from the image quality feedback system 100 in accordance with the embodiment of the second user terminal 400, as disclosed herein.

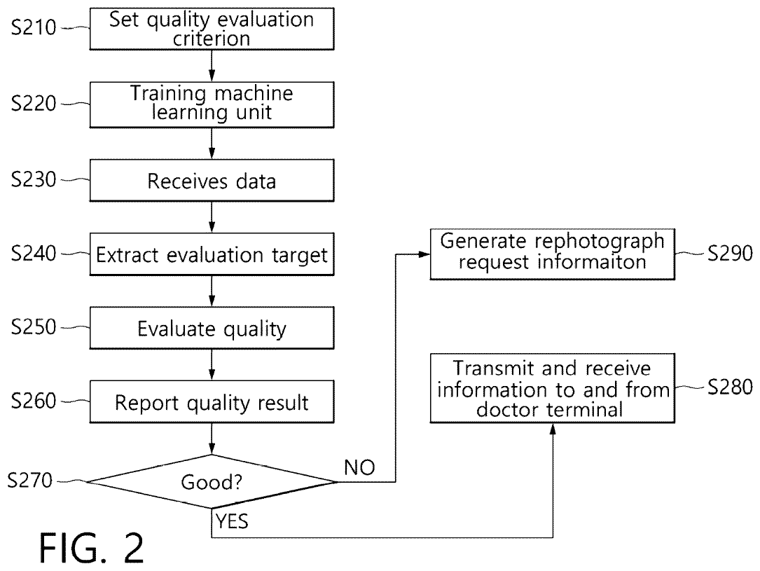

FIG. 2 shows a medical image quality feedback method according to an embodiment of the present disclosure.

Hereinafter, a medical image quality feedback method will be described based on the medical image quality feedback system 100. Unless otherwise specified, it may be understood that the medical image quality feedback method is performed by the image quality feedback system 100 or the controller 110 of the image quality feedback system 100.

Referring to FIG. 2, in step S210, the medical image quality feedback system 100 sets a quality evaluation criterion.

The quality evaluation criterion may be entered by the administrator or may be reflected in machine learning basic information in advance. For example, the quality evaluation criterion may be provided as machine learning basis data by a sample reflecting the quality evaluation according to the criterion.

The quality evaluation criterion may vary depending on the diagnosis method or policy of each hospital. For example, the quality evaluation criterion can be pre-defined by a healthcare provider or healthcare providing entity.

The quality evaluation criterion may vary depending on the analysis target. In an embodiment of the present disclosure, the lung area is illustrated as an analysis target, but it is not be limited thereto. For example, the present disclosure may be applied to X-ray quality evaluation for various parts such as diagnosis of spine and leg fracture, and the like.

In step S220, the controller 110 trains machine learning unit. In some examples, the controller 100 provides learning data to the first machine learning unit 112 and the second machine learning unit 114 to perform training about each problem-solving method.

The first machine learning unit 112 can learn how to extract the lung area by inputting the X-ray image.

The first machine learning unit 112 may output lung area extraction information for an arbitrary X-ray image by the learning.

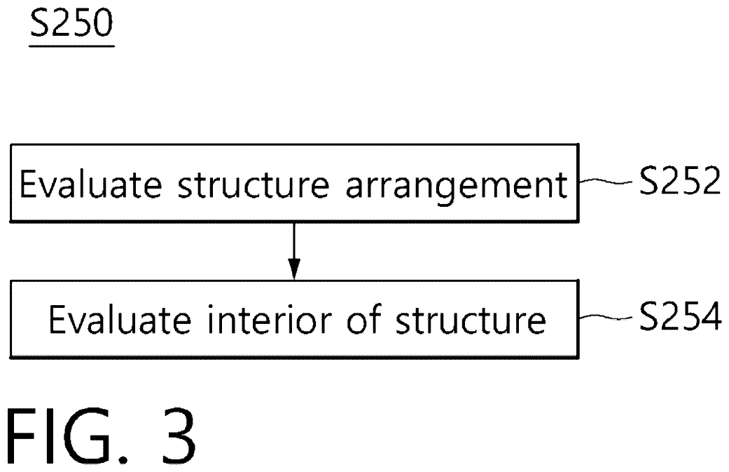

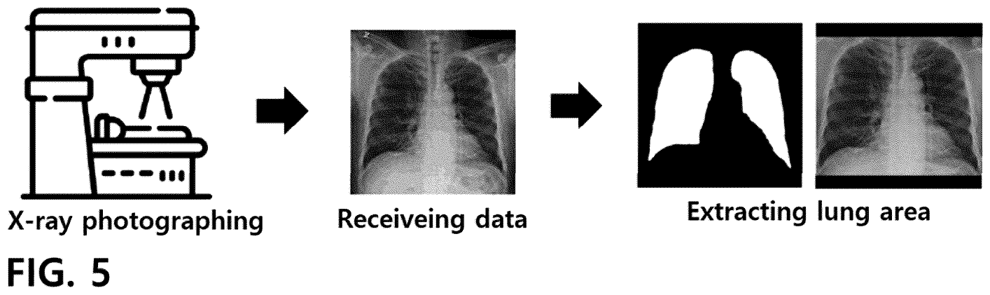

A process of extracting a lung area is illustrated in FIG. 5. For example, FIG. 3 shows a visualization of a process of extracting a lung area from a photographed X-ray image.

Further referring to FIG. 5, the captured X-ray image includes information on a lung, a spine, a rib, and an abdomen, but among them, the first machine learning unit 112 may separate or select and extract the area corresponding to the lung.

In some embodiments, the second machine learning unit 114 learns a relationship between the extracted lung area information and quality evaluation information.

In some embodiments, the second machine learning unit 114 outputs the quality evaluation information on arbitrary lung area information by the learning.

Referring back to FIG. 2, in step S230, the image quality feedback system 100 receives data from the X-ray image acquisition device 200.

For example, the received data may be an X-ray image. The X-ray image may include a lung area, but need not be limited thereto.

In step S240, the controller 110 extracts an evaluation target. Here, the controller 110 may specifically be the first machine learning unit 112.

The extracted target may be a region of interest to be diagnosed. In some examples, in an embodiment of the present disclosure, the evaluation target may be a lung area among the X-ray images.

The controller 110 may, for example, analyze an upper body X-ray image to separate or select a lung area and generate information.

Meanwhile, the extraction of the evaluation target is not necessarily performed by a module by machine learning. For example, the extraction of the evaluation target may be performed by a function that specifies a predetermined area as a relative position. For example, the extraction of the evaluation target may be extracted as information on a position specified using a statistical method.

In step S250, the controller 110 evaluates the quality of the extracted evaluation target. Here, the controller 110 may specifically be the second machine learning unit 114.

In some cases, the evaluation of the extracted target may not be performed by a module by machine learning. In some examples, the evaluation of the extracted target may be performed using a statistical analysis technique. As another example, the evaluation of the extracted target may be performed by using the statistical analysis technique and the machine learning method together.

For example, the quality evaluation of the lung area may include an evaluation of a structure arrangement and an evaluation of an interior of a structure.

FIG. 3 shows a detail process for perform step S250.

Referring to FIG. 3, in step S252, the controller 110 evaluates a structure arrangement.

The structure arrangement evaluation information may include an evaluation information of coverage range, an evaluation information of bilateral symmetry, and an evaluation information of scapular position.

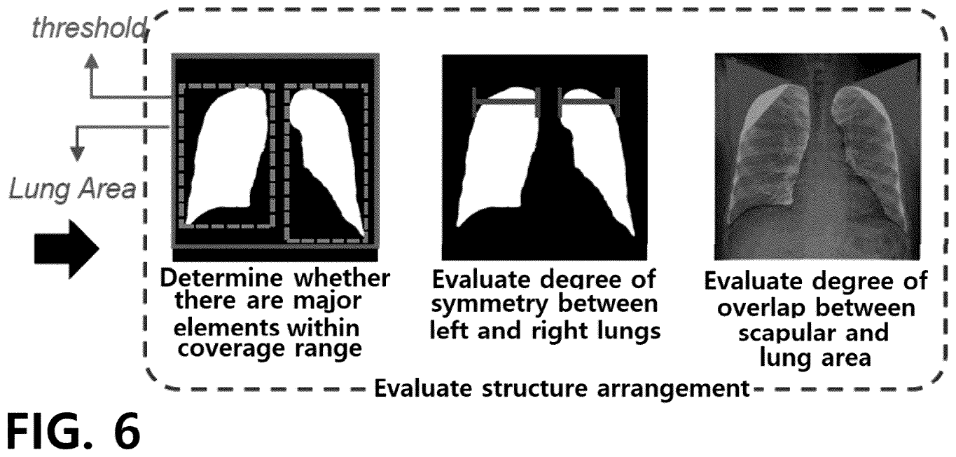

FIG. 6 shows visual representation of an embodiment of an evaluation of a structure arrangement.

In some embodiments, the structure may be defined as a component in the human body occupying a predetermined space or contour at a specific time. For example, the structure may include bones, a heart, lungs, and other organs.

Referring to FIG. 6, the evaluation of the coverage range is to determine whether there are major elements within the coverage range.

For example, when the first rib, the lateral diaphragm 3 cm or more downward, and both ends of the entire rib are identified in the image, it may be evaluated as satisfying the coverage criteria and not satisfying the other cases.

The evaluation of the bilateral symmetry is to evaluate the degree of symmetry between the left and right lungs.

The evaluation of the scapular position is to evaluate the degree of overlap between the scapular and the lung area.

When the degree of overlap between the scapula and the lung area is greater than a predetermined range, it may be evaluated as not satisfying the quality criterion, otherwise it may be evaluated as satisfying the quality criterion.

In step S254, the controller 110 is configured to evaluate an interior of the structure.

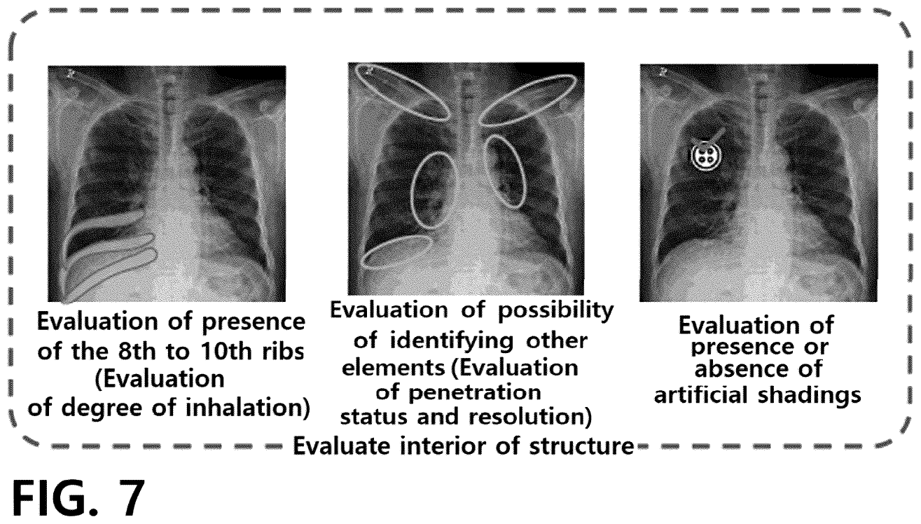

FIG. 7 shows visual representations of an embodiment of an evaluation of an interior of a structure.

Referring to FIG. 7, the evaluation information on the interior of the structure may include evaluation information on a degree of inhalation, evaluation information on a penetration state and a resolution, and evaluation information on a presence or absence of artificial shadings.

The evaluation of the degree of inhalation may be determined by whether the 8th to 10th ribs are identified on the image.

The evaluation of the penetration status and the resolution is to evaluate the observation of, for example, blood vessels in the outer ⅓ of the lung field, pulmonary blood vessels and a descending aorta behind the heart, blood vessels below the diaphragm, ribs above the diaphragm, diaphragm, intervertebral disc space, organs, and bronchus, and if only a portion of each is observed or is not clear, it may be evaluated as not meeting quality not meeting the quality criterion.

The evaluation of the penetration state and the resolution may be based on the case where a score, an average score, or some scores summed by scoring each element is less than a predetermined criterion. For example, the scores may be set in a manner such as 6 points for blood vessels clearly visible in the outer ⅓ of the lung area, 4 points for blood vessels visible in the outer ⅓ to ⅔ of the lung area, and 2 points for blood vessels visible only in the central ⅓ of the lung area.

The evaluation of the presence or absence of artificial shadings may be, for example, identifying a non-human body material, such as a button, projected onto the X-ray image.

Any identification of the non-human body material may be evaluated as not meeting the criterion for the presence or absence of artificial shadings.

In step S260, the controller 110 is configured to generate and report quality result information. The quality result information may be output through the indicator 130. The quality result information may be provided to the first user terminal 300. If the medical image quality feedback system 100 does not include the indicator 130, it may be effective to provide the quality result information to the first user terminal 300.

The quality result information may include X-ray image information and feedback information. The feedback information may include information on the reason when the quality criterion is not met, information on a correction request for a photographing posture corresponding to the above reason, and information on a rephotographing request.

The reason for not meeting the quality standard may include whether the criterion for evaluating a structure arrangement is met and whether the criterion for evaluating an interior of a structure is met.

The quality result information may be output in an audiovisual manner. Therefore, the present disclosure may further include outputting the quality result information in an audiovisual manner.

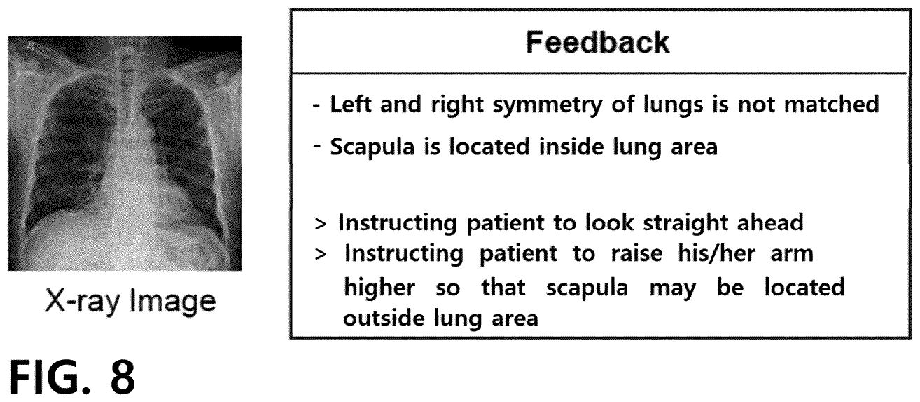

FIG. 8 shows both the X-ray image information and feedback information according to an embodiment of the present disclosure.

The X-ray image on the left side of FIG. 8 may be the same image as the image received from the X-ray image acquisition device 200.

The right side of FIG. 8 includes feedback information. In the embodiment, the feedback information may include a reason for not meeting the quality standard, such as “left and right symmetry of the lungs is not matched, the scapula is located inside the lung area.

The feedback information may include information on the request for correcting the photography posture, such as “instructing the patient to look straight ahead”, “instructing the patient to raise his/her arm higher so that the scapula may be located outside the lung area”.

The feedback information may simply include information indicating “re-photography need.”

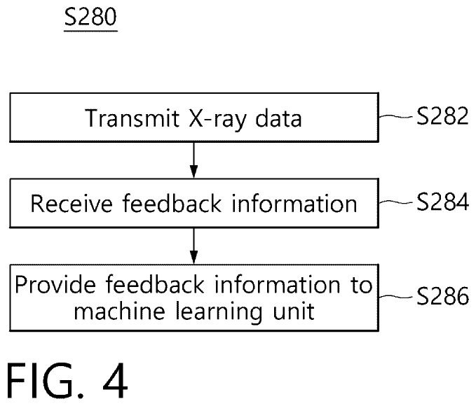

FIG. 4 shows an detail process of a medical image quality feedback method according to an embodiment of the present disclosure.

Referring to FIG. 4, in step S282, the medical image quality feedback system 100 transmits X-ray data to the second user terminal 400.

Here, the X-ray data transmitted to the second user terminal 400 may be determined as data to be transmitted when the evaluated X-ray data in step S250 satisfies a predetermined quality criterion.

In step S284, the medical image quality feedback system 100 receives expert feedback information from the second user terminal 400.

The expert feedback information is information that experts, who are doctors, directly judge whether quality standards are met compared to the machine learning unit and describes the reason for not satisfying the quality criterion when the quality standard is not satisfied. For example, the expert feedback information may include information of “The 9th rib is not well identified. There is a need to take a larger breath and photograph.”

In step S286, the medical image quality feedback system 100 provides the expert feedback information to the second machine learning unit 114.

The second machine learning unit 114 may input the expert feedback information as the additional learning information and reflect it in subsequent quality evaluations.

The expert feedback information may be information evaluated based on criteria different from the above-described predetermined quality criterion. Since there may be individual differences for each expert, the medical image quality feedback system 100 may separately generate individual learning fields (classes) for each expert and reflect it in machine learning. As a result, the second machine learning unit 114 may output the learning results reflected not only the predetermined quality criterion but also the individually fed-back requirements of the expert as a result of the quality evaluation.

The terminology used in the present application is used merely to describe specific embodiments, and is not intended to limit the present disclosure. In this application, it should be understood that the terms “to include” or “to have” are intended to designate the presence of features, numbers, steps, operations, elements, components, or combinations thereof described in the specification, but do not preclude the presence or possibility of addition of one or more other features, numbers, steps, operations, elements, components, or combinations thereof.

Logical blocks, visual representations, modules or units described in connection with embodiments disclosed herein can be implemented or performed by a computing device having at least one processor, at least one memory and at least one communication interface. The elements of a method, process, or algorithm described in connection with embodiments disclosed herein can be embodied directly in hardware, in a software module executed by at least one processor, or in a combination of the two. Computer-executable instructions for implementing a method, process, or algorithm described in connection with embodiments disclosed herein can be stored in a non-transitory computer readable storage medium.

What is claimed is:

1. A method for feeding back medical image quality, wherein the method is performed by at least one processor, comprising:
receiving X-ray image data;
extracting a region of interest to be diagnosed from the image data;
evaluating a quality of the region of interest depending on a predetermined criterion; and
generating feedback information including whether to request rephotographing based on a result of the quality evaluation.

2. The method of claim 1, further comprising: outputting the feedback information in an audiovisual manner,
wherein the feedback information includes a reason for not meeting the quality criterion.

3. The method of claim 2, wherein the reason for not meeting the quality standard includes at least one of:
the criterion for evaluating a structure arrangement being met and
the criterion for evaluating an interior of a structure being met.

4. The method of claim 3, wherein the evaluation of the structure arrangement includes an evaluation of at least one of:
an evaluation of coverage range,
an evaluation of bilateral symmetry, and
an evaluation of scapular position.

5. The method of claim 3, wherein the evaluation of the interior of the structure includes an evaluation of at least one of:
an evaluation of a degree of inhalation,
an evaluation of a penetration state and a resolution, and
an evaluation of a presence or absence of artificial shadings.

6. The method of claim 1, further comprising:
setting a criterion of the quality evaluation; and
determining learning data depending on the quality evaluation criterion and performing machine learning,
wherein the predetermined criterion is the quality evaluation criterion,
wherein the quality evaluation is output by the machine-learned result,
wherein the region of interest is a lung area, and
wherein the quality evaluation includes at least one of:
an evaluation of a structure arrangement and
an evaluation of an interior of a structure.

7. The method of claim 1, further comprising:
setting a criterion of the quality evaluation;
determining learning data depending on the quality evaluation criterion and performing machine learning;
determining the X-ray image as an image to be transmitted to an expert and transmitting it to an expert terminal;
receiving expert feedback information from the expert terminal; and
determining the expert feedback information as additional learning data and performing machine learning,
wherein the quality evaluation is output by the machine-learned result.

8. A system for feeding back medical image quality comprising:
a communicator configured to receive X-ray image data; and
a processor configured to:
extract a region of interest to be diagnosed from the image data,
evaluate a quality of the region of interest depending on a predetermined criterion, and
generate feedback information including whether to request rephotographing based on a result of the quality evaluation.
